(19) United States
(12) Patent Application Publication
(10) Pub. No.: US 2005/0209162 A1
(43) Pub. Date: Sep. 22, 2005

(54) Methods for monitoring IL-18

(76) Inventors: Amit Roy, Plainsboro, NJ (US); Matthew W. Harding, Acton, MA (US); John Alam, Cambridge, MA (US); John Randle, Brookline, MA (US)

Correspondence Address: FISH & NEAVE IP GROUP;ROPES & GRAY LLP, 1251 AVENUE OF THE AMERICAS FL C3, NEW YORK, NY, 10020-1105, US

(21) Appl. No.: 10/985,641

(22) Filed: Nov. 10, 2004

Related U.S. Application Data
(60) Provisional application No. 60/519,055, filed on Nov. 10, 2003.

(51) Int. Cl. A61K 38/04; G01N 33/00

(52) U.S. Cl. 514/19; 436/86

(57) ABSTRACT

This invention relates to methods for monitoring IL-18.

### CROSS-REFERENCE TO RELATED APPLICATION(S)

This application claims the benefit under 35 U.S.C. § 119 of U.S. Provisional Application 60/519,055, filed Nov. 10, 2003, the entire contents of that application being incorporated herein by reference Field of the Invention

### FIELD OF THE INVENTION

This invention relates to methods for monitoring IL-18.

### BACKGROUND OF THE INVENTION

Interleukin-1β converting enzyme (ICE), also termed caspase-1, is the cysteine protease primarily responsible for cleaving pro-interleukin-1β (pro-IL-1β) and pro-interleukin-18 (pro-IL-18) into their biologically active forms (IL-1β and IL-18). (Nakanishi K, Y. T., Tsutsui H, Okamura H., Interleukin-18 regulates both Th1 and Th2 responses. Annu Rev Immunol. 2001. 19:423-474; Dinarello, C., Biologic basis for interleukin-1 in disease. Blood 1996; 87(6):2095-2147). Following this conversion to biologically active forms, IL-1β and IL-18 are released from cells to mediate their functions. ICE is constitutively expressed in macrophages, T lymphocytes, and neutrophils. ICE expression is also induced under certain conditions in other cell types such as keratinocytes.

IL-1β and IL-18 have important roles in acute and chronic inflammatory immune responses. IL-1β induces the expression of several mediators of immune cell response including the pro-inflammatory cytokines tumour necrosis factor α (TNFα) and IL-6, cyclooxygenase-2 (COX-2), chemokines and cell surface adhesion molecules that target cells to a site of infection or injury. (Dinarello, C., Biologic basis for interleukin-1 in disease. Blood 1996; 87(6):2095-2147; Fantuzzi, G., Lessons from interleukin-deficient mice: the interleukin-1 system. Acta Physiol Scand 2001; 173(1):5-9). IL-18 also induces chemokine and adhesion molecule expression. In addition, IL-18 acts in synergy with IL-12 to induce the production of IFN-γ by Type 1 T lymphocytes and activates natural killer (NK) cells, each characteristic of a cell-mediated immune response resulting from infection with a pathogen or other inflammatory stimulus. IL-1β and IL-18 also have roles in parenchymal tissues. For example, IL-1β and IL-18 modulate bone metabolism by osteoclasts, and IL-18 is produced by keratinocytes and other cell types. (Dinarello, C., Biologic basis for interleukin-1 in disease. Blood 1996; 87(6):2095-2147; Ambramson, S. B. and Amin A, Blocking the effects of IL-1 in rheumatoid arthritis protects bone and cartilage. Rheumatology 2002; 41:972-80; Udagawa, N, Horwood N J, Elliott J, Mackay A, Owens J, Okamura H, Kurimoto M, Chambers T J, Martin T J, Gillepsie M T, Interleukin-18 (interferon-γ-inducing factor) is produced by osteoblasts and acts via granulocyte/macrophage colony-stimulating factor and not via interferon-g to inhibit osteoclast formation. J Exp Med 1997; 185(6); 1005-12; Naik S M, C. G., Burbach G J, Singh S R, Swerlick R A, Wilcox J N, Ansel J C, Caughman S W, Human keratinocytes constitutively express interleukin-18 and secrete biologically active interleukin-18 after treatment with pro-inflammatory mediators and dinitrochlorobenzene. J Invest Dermatol 1999; 113:766-772).

The generation of ICE-deficient mice provided a murine model system in which to evaluate the physiopathological roles of ICE and predict the safety and efficacy of ICE inhibition as a therapeutic strategy. ICE-deficient mice respond to many inflammatory stimuli with markedly decreased production of IL-1β, confirming that ICE is the primary enzyme responsible for the cleavage of pro-IL-1β. (Kuida K, L. J., Ku G, Harding M W, Livingston D J, Su M S, Flavell R A, Altered cytokine export and apoptosis in mice deficient in interleukin-1 beta converting enzyme. Science 1995; 267(5206):2000-2003; Li P, A. H., Banerjee S, Franklin S, Herzog L, Johnston C, McDowell J, Paskind M, Rodman L, Salfeld J, et al., Mice deficient in IL-1 beta-converting enzyme are defective in production of mature IL-1 beta and resistant to endotoxic shock. Cell 1995; 80(3):401-411). IL-18 production is less profoundly inhibited in ICE-deficient mice, indicating that in addition to ICE, other proteolytic enzymes may also process pro-IL-18. Further characterization of ICE-deficient mice has shown that in certain instances other proteases, while not in the predominant processing route, are able to cleave pro-IL-1β and pro-IL-18 to their biologically active forms. (Nakanishi K, Y. T., Tsutsui H, Okamura H., Interleukin-18 regulates both Th1 and Th2 responses. Annu Rev Immunol. 2001. 19:423-474; Fantuzzi G, Harding M W, Livingston D J, Sipe J D, Kuida K, Flavell R A, Dinarello C A, Response to local inflammation of IL-1 beta-converting enzyme-deficient mice. J Immunol 1997; 158(4): 1818-1824).

IL-1β and IL-18, in the presence of IL-12, promote an immune response characterized by production of IFNγ. This cytokine profile is typical of a Type 1 immune response associated with cell-mediated immunity primarily in response to pathogens or other inflammatory signals, which is an appropriate response to these stimuli. However a Type 1 response can be detrimental when initiated inappropriately or prolonged, as evidenced by certain diseases such as psoriasis, Crohn's disease, multiple sclerosis, and rheumatoid arthritis. (Nakanishi K, Y. T., Tsutsui H, Okamura H., Interleukin-18 regulates both Th1 and Th2 responses. Annu Rev Immunol. 2001. 19:423-474; Dinarello, C., Biologic basis for interleukin-1 in disease. Blood 1996; 87(6):2095-2147; (Singh B, P. F., Mortensen N J, Immune therapy in inflammatory bowel disease and models of colitis. Br J Surg 2001; 88(12):1558-1569; Abramson S B, A. A., Blocking the effects of IL-1 in rheumatoid arthritis protects bone and cartilage. Rheumatology 2002; 41(9):972-980; Ohta Y, H. Y., Katsuoka K, Expression of IL-18 in psoriasis. Arch Dermatol Res 2001; 293(7):334-342).

Psoriasis is an inflammatory disease of the skin characterized by scaly, red, and indurated lesions varying in size and extent of affected body surface area. [Krueger G G, F. S., Camisa C, Duvic M, Elder J T, Gottlieb A B, Koo J, Krueger J G, Lebwohl M, Lowe N, Menter A, Morison W L, Prystowsky J H, Shupack J L, Taylor J R, Weinstein G D, Barton T L, Rolstad T, Day R M, “Two considerations for patients with psoriasis and their clinicians: what defines mild, moderate, and severe psoriasis? What constitutes a clinically significant improvement when treating psoriasis?” J Am Acad Dermatol, 43, pp. 281-285 (2000)]. Immunohistochemical analysis of human psoriatic lesions has revealed the infiltration of both CD4+ and CD8+ T lymphocytes, which predominantly express IFN-γ and TNFα˜. [(Krueger, J G “The immunologic basis for the treatment of psoriasis with new biologic agents” J Am Acad Dermatol, 46, pp. 1-23 (2002)]. When the skin is infected or irritated, Langerhans cells (LCs), skin specific antigen presenting cells that express IL-12, migrate to draining lymph nodes resulting in the homing of T lymphocytes to the specific site of infection or irritation. The migration of LCs is dependent upon IL-18 and IL-1β and blocking of either cytokine prevents the migration of LCs to the draining lymph nodes. [Cumberbatch M, D. R., Antonopoulos C, Groves R W, Kimber I, “Interleukin (IL)-18 induces Langerhans cell migration by a tumour necrosis factor-alpha- and IL-1beta-dependent mechanism” Immunology, 102, pp. 323-330 (2001).] Keratinocytes, an integral cell type in the differentiation of the epidermis, constitutively express pro-IL-1β and pro-IL-18 but under normal circumstances do not express ICE. (Naik S M, C. G., Burbach G J, Singh S R, Swerlick R A, Wilcox J N, Ansel J C, Caughman S W, Human keratinocytes constitutively express interleukin-18 and secrete biologically active interleukin-18 after treatment with pro-inflammatory mediators and dinitrochlorobenzene. J Invest Dermatol 1999; 113:766-772). Expression of ICE is induced in keratinocytes by contact sensitizing agents, such as dinitrochlorobenzene. (Cumberbatch M, D. R., Antonopoulos C, Groves R W, Kimber I, Interleukin (IL)-18 induces Langerhans cell migration by a tumour necrosis factor-alpha- and IL-1beta-dependent mechanism. Immunology 2001; 102:323-330; Zepter K, A. Haffner, L. F. Soohoo, D. De Luca, H. P. Tang, P. Fisher, J. Chavinson, and C. A. Elmets, Induction of biologically active IL-1-β-converting enzyme and mature IL-1b in human keratinocytes by inflammatory and immunologic stimuli. J. Immunol. 1997; 159:6203-8). The significance of ICE expression in keratinocytes was evaluated in transgenic mice engineered to constitutively express ICE in keratinocytes. (Yamanaka K, T. M., Tsutsui H, Kupper T S, Asahi K, Okamura H, Nakanishi K, Suzuki M, Kayagaki N, Black R A, Miller D K, Nakashima K, Shimizu M, Mizutani H, Skin-specific caspase-1-transgenic mice show cutaneous apoptosis and pre-endotoxin shock condition with a high serum level of IL-18. J Immunol 2000; 165(2):997-1003). At 8 weeks of age, these mice develop chronic active dermatitis surrounding the eyes, of the face, ear, neck, trunk, and legs. While the lesions can heal intermittently, the erosion and ulceration of the skin relapses. The histological changes resemble that of human psoriatic lesions. The importance of inflammatory cytokines in psoriasis has been further validated in clinical trials of anti-TNFα biologic therapies, such as etanercept (soluble TNF-α receptor) and infliximab (humanized anti-TNFa monoclonal antibody), which indicate a clinical benefit. (Mease P J, G. B., Metz J, VanderStoep A, Finck B, Burge D J, Etanercept in the treatment of psoriatic arthritis and psoriasis: a randomised trial. Lancet 2000; 356(9227): 385-390; Chaudhari U, R. P., Mulcahy L D, Dooley L T, Baker D G, Gottlieb A B, Efficacy and safety of infliximab monotherapy for plaque-type psoriasis: a randomised trial. Lancet 2001; 357:1842-1847).

The significance of inhibiting inflammatory cytokines in rheumatoid arthritis has been highlighted by the approved parenteral, biological therapies infliximab and adalimumab (humanized anti-TNF-α monoclonal antibodies), etanercept (soluble TNF-α receptor), and anakinra (soluble IL-1 receptor antagonist). Inhibition of TNF-α has also shown utility in Crohn's disease patients treated with infliximab, an approved therapy. Proof-of-mechanism for ICE inhibition in rheumatoid arthritis has been shown in a Phase II study with an investigational ICE inhibitor, pralnacasan. ((Pavelka K, Kuba V, Moeller Rasmussen J, Mikkelsen K, Tamasi L, Vitek P, Rozman B. Clinical effects of pralnacasan (PRAL), an orally-active interleukin-1b converting enzyme (ICE) inhibitor, in a 285 patient Ph II trial in rheumatoid arthritis (RA). [American College of Rheumatology 2002 Conference; Late-Breaking Abstract; New Orleans, La., USA].

Nevertheless, developing therapeutic uses for ICE inhibitors has been hindered by a lack of clinical benchmarks. For example, levels of IL-1 in plasma are typically too low to measure by ELISA assays. Thus, it can be difficult to determine whether administration of a compound for inhibiting IL-1β, such as an ICE inhibitor, is having an effect in vivo. There is, therefore, a need for a biomarker relevant to administration of an ICE inhibitor and other potentially biologically active compounds. There is also a need for potent, orally active compounds that inhibit IL-18.

### SUMMARY OF THE INVENTION

The present invention relates to methods for measuring IL-18 levels. The invention provides methods for measuring IL-18 in human fluids for the purpose of diagnosing disease and evaluating of the response to ICE inhibitor or other therapy. These method are useful in discovering or developing compounds that modulate IL-18.

### BRIEF DESCRIPTION OF THE FIGURES

FIG. 1 depicts (median+/−standard deviation) serum IL-18 levels from groups of 9 subjects treated with Compound A (900 mg q8 h; filled circles) or placebo (q8 h; open circles) during 14 days.

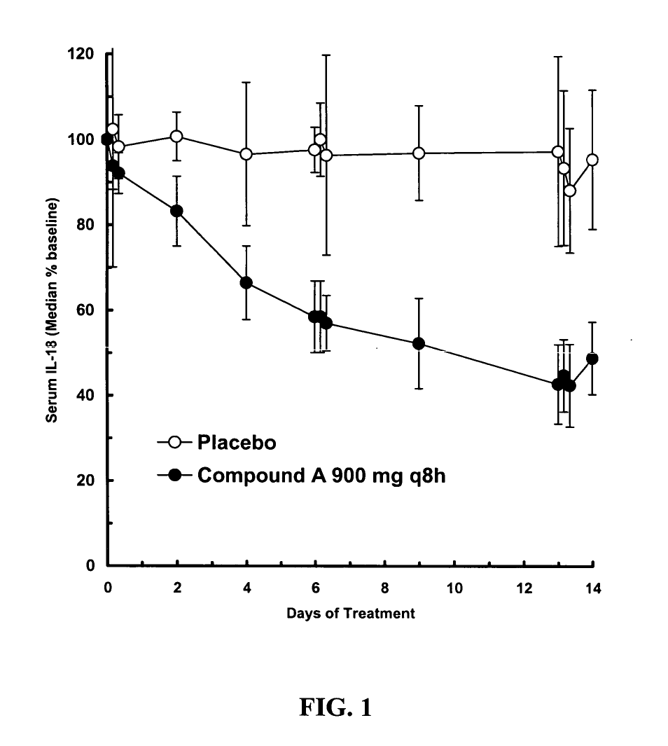

### DETAILED DESCRIPTION OF THE INVENTION

This invention provides methods for monitoring IL-18, primarily in clinical settings.

Monitoring IL-18 levels provides a method for monitoring both IL-18-mediated and IL-1 β mediated disease processes. Additionally, monitoring IL-18 levels provides an advantage in monitoring IL-1β mediated disease treatment, as IL-1β is not consistently elevated in many disease states. Other potential biomarkers also appear to be inadequate for monitoring IL-18-mediated and IL-1 β-mediated biological events [Konstan, M. W. et al., “Effect of Ibuprofen on Neutrophil Migration In Vivo in Cystic Fibrosis and Healthy Subjects” The Journal of Pharmacology and Experimental Therapeutics, 306, pp. 1086-1091 (2003). Both IL-18 and IL-1β are processed by the same enzyme, ICE (caspase-1). Therefore, monitoring IL-18 modulation is a “proxy” for monitoring IL-1β modulation.

Additionally, IL-18 may be used as an early response indicator for, e.g., the effectiveness of a drug. By using IL-18 as a biomarker, the presence of what would be an ultimately positive response (i.e., a delayed response) to a drug could be appreciated sooner.

Assays for both inactive IL-18 (proIL-18) and active IL-18 are available. Assays for monitoring active IL-18 should be used in the methods of this invention (see Examples; K. Shida et al, “An Alternative Form of IL-18 in Human Blood Plasma: Complex Formation with IgM Defined by Monoclonal Antibodies”, J. Immunol., 166, pp. 6671-6679 (2001); and M. Taniguchi, “Characterization of Anti-human Interleukin-18 (IL-18)/Interferon-γ-inducing Factor (IGIF) Monoclonal Antibodies and their Application in the Measurement of Human IL-18 by ELISA”, J. Immunol. Methods, 206, pp. 107-113 (1997)). Any such assay could be used in connection with this invention (see, Interleukin-18, GlaxoSmithKline Clinical Data, R&D Focus Drug News, Jun. 23, 2003). Samples for conducting the IL-18 measurements may be obtained from any biological source including, but not limited to, serum, blood, and tissue.

An ICE inhibitor of this invention has been tested as described above and shown to inhibit IL-18. Compound A reduced serum IL-18 median levels gradually over 14 days of treatment, reaching approximately 60% inhibition. Treatment with placebo did not modify serum IL-18 median levels.

Without being bound by theory, Compound A is thought to be a prodrug of Compound B. Compound A is rapidly absorbed upon oral administration and converted into Compound B. Compound B is a selective and reversible ICE inhibitor. The ICE inhibition constant (Ki) for Compound B is 0.8 nM.

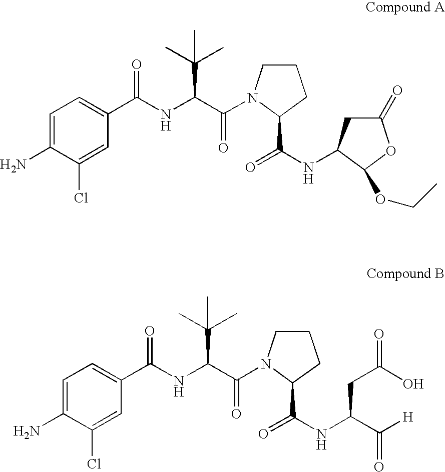

This certain embodiments, this invention provides for comparing the IL-18 levels in a subject before and after treatment with a therapeutic compound or other therapy.

Accordingly, in one embodiment is provided a method for evaluating whether an ICE inhibitor is effective in ameliorating, treating or preventing an IL-1 mediated condition and/or an IL-18 mediated condition, the method comprising:
measuring IL-18 levels in the blood of patients before treatment,
and further measuring IL-18 levels in the blood of patients after treatment,
   wherein a decrease in IL-18 levels after treatment is indicative of effectiveness.

This invention also provides a method for evaluating the pharmacodynamics of compounds by monitoring IL-18 levels in vivo. Advantageously, testing for IL-18 modulation is essentially a way to test the pharmacodynamics of a compound. Most methods (e.g., assay methods) would evaluate the pharmacokinetics (i.e., the effect of the body on the drug) of a compound. [Rowland, M. and Tozer, T. N., Clinical pharmacokinetics: Concepts and applications, 3^{rd} Ed., Lippincott Williams & Wilkins, Philadelphia, (1995); Yu, D. K., Bhargava V. O., and Weir S. J., “Selection of doses for phase II clinical trials based on pharmacokinetic variability consideration. J Clin Pharmacol. 37(8), pp. 673-8 (1997).] Pharmacokinetics measures the effect of the body on the drug, whereas pharmacodynamics measures the effect of the drug on the body. In drug discovery, a method for measuring the pharmacodynamics of a drug would, in many cases, be more useful than measuring the pharmacokinetics of the drug.

Methods for evaluating the pharmacodynamics of compounds are useful in many aspects of drug discovery and drug development. The methods may be used, for example, to evaluate compounds, pharmaceutical compositions, formulations, dosage forms, dosages, and dosing and therapeutic regimens (including dose level, dose administration route, and dose frequency), either alone or in combination.

This invention also provides for evaluating a compound formulation. Accordingly, another embodiment provides a method for evaluating whether a formulation comprising an ICE inhibitor is effective in ameliorating, treating or preventing an IL-1 mediated condition and/or an IL-18 mediated condition, the method comprising:
measuring IL-18 levels in the blood of patients before treatment with said formulation,
and further measuring IL-18 levels in the blood of patients after treatment with said formulation,
wherein a decrease in IL-18 levels after treatment is indicative of effectiveness.

This invention also provides for evaluating a dose of a drug or a dosage regimen of a drug. Accordingly, another embodiment provides a method for evaluating whether a dosage amount or regime of an ICE inhibitor is effective in ameliorating, treating or preventing an IL-1 mediated condition and/or an IL-18 mediated condition, the method comprising:
measuring IL-18 levels in the blood of patients before treatment with said dosage amount or regime, and further measuring IL-18 levels in the blood of patients after treatment with said dosage amount or regime,
wherein a decrease in IL-18 levels after treatment is indicative of effectiveness.

Methods of this invention are useful in clinical trials, in evaluating the efficacy of a therapeutic regimen, or in monitoring treatment of a subject. Subjects include animals, such as primates, rodents, and birds, (guinea pigs, hamsters, gerbils, rat, mice, rabbits, dogs, cats, horses, pigs, sheep, cows, goats, rhesus monkeys, monkeys, tamarinds, apes, baboons, gorillas, chimpanzees, orangutans, gibbons, chickens, turkeys, ducks, and geese). Zoo, laboratory, and farm animals could be subjects under this invention. A preferred subject is a mammal and is more preferably a human.

In another embodiment, this invention provides a method of determining whether a patient is a candidate for therapy with an ICE inhibitor, comprising determining IL-18 levels in the subject, comparing IL-18 levels in the subject with IL-18 levels in a normal individual, wherein higher IL-18 levels in the potential subject qualifies the patient for therapy.

In yet another embodiment, this invention provides a method for predicting the therapeutic outcome of an ICE inhibitor therapy, comprising determining IL-18 levels in the subject, prior to and after administration of the ICE inhibitor, wherein a decrease in IL-18 levels after administration of the ICE inhibitor is predictive of a potentially successful therapeutic outcome.

Also provided by this invention is a method of following the course of therapy with an ICE inhibitor comprising the step of monitoring the levels of IL-18 in the patient at the beginning and during continuation of therapy.

Any compound may be tested in the assays of this invention. Compounds where modulation of IL-18 is of interest may be tested. Preferred compounds are those wherein decrease of IL-18 is therapeutically useful. However, nothing limits the methods of this invention being used to monitor, for example, side-effects involving IL-18 modulation.

For example, a compound that inhibits IL-18, by, e.g., neutralizing IL-18 activity, such as IL-18BP (WO 99/09063, or an anti-IL-18 antibody may be tested as disclosed herein.

ICE inhibitors are a class of compounds that may be used and/or tested in the methods of this invention. Any compound that inhibits ICE may be used in the methods and compositions of this invention. Such compounds include those compounds that inhibit ICE selectively and those that inhibit one or more enzyme in the caspase or ICE/CED-3 family. Examples of compounds that may be tested according to this invention include, but are not limited to, the compounds described in WO 04/058718, WO 04/002961, WO 03/088917, WO 03/068242, WO 03/042169, WO 98/16505, WO 93/09135, WO 00/55114, WO 00/55127, WO 00/61542, WO 01/05772, WO 01/10383, WO 01/16093, WO 01/42216, WO 01/72707, WO 01/90070, WO 01/94351, WO 02/094263, WO 02/42278, WO 03/106460, WO 03/103677, WO 03/104231, U.S. Pat. No. 6,184,210, U.S. Pat. No. 6,184,244, U.S. Pat. No. 6,187,771, U.S. Pat. No. 6,197,750, U.S. Pat. No. 6,242,422, April 2001 American Chemical Society (ACS) meeting in San Diego, Calif., USA., WO 02/22611, U.S. 2002/0058630, WO 02/085899, WO 95/35308, WO 97/22619, WO 99/47545, and WO 01/90063. Preferred compounds for testing according to this invention are described in WO 04/058718, WO 04/002961, WO 95/35308, WO 97/22619, WO 99/47545, and WO 01/90063.

Included would be all isomeric (e.g., enantiomeric, diastereomeric, and geometric (or conformational)) forms of the structures; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the cited structure except for the replacement of hydrogen by deuterium or tritium, or the replacement of a carbon by a ¹³C- or ¹⁴C-enriched carbon are within the scope of this invention.

According to another embodiment, this invention provides a method for identifying a compound that ameliorates, treats, or prevents an IL-1 mediated disease, the method comprising:
measuring IL-18 levels in a subject prior to administration of the compound,
and further, measuring the levels after administration of the compound,
wherein a decrease in the IL-18 levels after administration of the compound indicates the compound may ameliorate, treat, or prevent the IL-1 mediated condition or disease.

According to yet another embodiment, this invention provides a method for identifying a compound that ameliorates, treats, or prevents an IL-18 mediated disease, the method comprising:
measuring IL-18 levels in a subject prior to administration of the compound,
and further, measuring the levels after administration of the compound,
   wherein a decrease in the IL-18 levels after administration of the compound indicates the compound may ameliorate, treat, or prevent the IL-18 mediated condition or disease.

Applicants have demonstrated inhibition of IL-18 in vivo in humans and animals with an ICE inhibitor. Accordingly, another embodiment of this invention provides methods for inhibiting IL-18 by administering a compound and monitoring the IL-18 inhibition according to the methods of this invention.

Compounds of this invention may be tested for their ability to inhibit ICE and decrease IL-18 levels. In addition to testing in the methods of this invention, these compounds can be assayed, for example, for their ability to inhibit the production of IL-1β, and/or regulate IL-1β levels and/or IL-1β activity. Assays for each of the activities are known in the art, including those described herein.

This invention also provides a composition comprising a compound selected or evaluated according to a method of this invention or a pharmaceutically acceptable derivative (e.g., salt) thereof, and a pharmaceutically acceptable carrier.

The pharmaceutical compositions and methods of this invention, therefore, will be useful for controlling IL-1β levels and/or activity in vitro or in vivo. The compositions and methods of this invention will thus be useful for controlling IL-18 or IL-1β levels in vivo and for ameliorating, treating, preventing, or reducing the advancement, severity or in effects of certain conditions, including diseases, disorders, or effects as set forth herein.

Pharmaceutical compositions are well known in the art (Ainley Wade and Paul J Weller, Handbook of Pharmaceutical Excipients, second edition, American Pharmaceutical Association, 2215 Constitution Avenue, NW, Washington D.C. 20037-2985 USA, and the Pharmaceutical Press, Royal Pharmaceutical Society of Great Britain, 1 Lambeth High Street, London, SE1 7JN, England). Certain pharmaceutical compositions are also described herein.

According to another embodiment, the compositions of this invention may further comprise another therapeutic agent. Such agents include, but are not limited to, a thrombolytic agent such as tissue plasminogen activator and streptokinase, an anti-inflammatory agent, a matrix metalloprotease inhibitor, a lipoxygenase inhibitor, a cytokine antagonist, a cytokine inhibitor, a cytokine antibody, a cytokine binding protein, an immunosuppressant, an anti-cancer agent, an anti-viral agent, a cytokine, a growth factor, an immunomodulator (e.g., bropirimine, anti-human alpha interferon antibody, IL-2, GM-CSF, methionine enkephalin, interferon alpha, diethyldithiocarbamate, tumor necrosis factor (TNF), a TNF inhibitor, naltrexone and rEPO), a prostaglandin, or an anti-vascular hyperproliferation compound.

The term “pharmaceutically acceptable carrier” refers to a non-toxic carrier that may be administered to a patient, together with a compound of this invention, and which does not destroy the pharmacological activity thereof.

Pharmaceutically acceptable carriers that may be used in these compositions include, but are not limited to, ion exchangers, alumina, aluminum stearate, lecithin, serum proteins such as human serum albumin, buffer substances such as phosphates, glycine, sorbic acid, potassium sorbate, partial glyceride mixtures of saturated vegetable fatty acids, water, salts or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

In pharmaceutical compositions comprising only a compound of this invention as the active component, methods for administering these compositions may additionally comprise the step of administering to the subject an additional agent. Such agents include, but are not limited to, a thrombolytic agent such as tissue plasminogen activator and streptokinase, an anti-inflammatory agent, a matrix metalloprotease inhibitor, a lipoxygenase inhibitor, a cytokine antagonist, a cytokine inhibitor, a cytokine antibody, a cytokine binding protein, an immunosuppressant, an anti-cancer agent, an anti-viral agent, a cytokine, a growth factor, an immunomodulator (e.g., bropirimine, anti-human alpha interferon antibody, IL-2, GM-CSF, methionine enkephalin, interferon alpha, diethyldithiocarbamate, tumor necrosis factor (TNF), a TNF inhibitor, naltrexone and rEPO), a prostaglandin, or an anti-vascular hyperproliferation compound. When a second agent is used, the second agent may be administered either as a separate dosage form or as part of a single dosage form with the compounds or compositions of this invention.

The amount of compound present in the above-described compositions should be sufficient to cause a detectable decrease in the severity of the disease, or in ICE inhibition, IL-1β levels, or IL-1 activity.

If pharmaceutically acceptable salts of the compounds of this invention are utilized in these compositions, those salts are preferably derived from inorganic or organic acids and bases. Included among such acid salts are the following: acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate and undecanoate. Base salts include ammonium salts, alkali metal salts, such as sodium and potassium salts, alkaline earth metal salts, such as calcium and magnesium salts, salts with organic bases, such as dicyclohexylamine salts, N-methyl-D-glucamine, and salts with amino acids such as arginine, lysine, and so forth.

Also, the basic nitrogen-containing groups can be quaternized with such agents as lower alkyl halides, such as methyl, ethyl, propyl, and butyl chlorides, bromides and iodides; dialkyl sulfates, such as dimethyl, diethyl, dibutyl and diamyl sulfates; long chain halides such as decyl, lauryl, myristyl and stearyl chlorides, bromides and iodides; aralkyl halides, such as benzyl and phenethyl bromides and others. Water or oil-soluble or dispersible products are thereby obtained.

The compounds utilized in the compositions and methods of this invention may also be modified by appending appropriate functionalities to enhance selective biological properties. Such modifications are known in the art and include those which increase biological penetration into a given biological system (e.g., blood, lymphatic system, or central nervous system), increase oral availability, increase solubility to allow administration by injection, alter metabolism and/or alter rate of excretion.

According to a preferred embodiment, the compositions of this invention are formulated for pharmaceutical administration to a subject, e.g., a mammal, preferably a human being.

Such pharmaceutical compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection and infusion techniques. Preferably, the compositions are administered orally or intravenously.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil and castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

If a solid carrier is used, the preparation can be tableted, placed in a hard gelatin capsule in powder or pellet form, or in the form of a troche or lozenge. The amount of solid carrier will vary, e.g., from about 25 mg to 400 mg. When a liquid carrier is used, the preparation can be, e.g., in the form of a syrup, emulsion, soft gelatin capsule, sterile injectable liquid such as an ampule or nonaqueous liquid suspension. Where the composition is in the form of a capsule, any routine encapsulation is suitable, for example, using the aforementioned carriers in a hard gelatin capsule shell.

A syrup formulation can consist of a suspension or solution of the compound in a liquid carrier for example, ethanol, glycerin, or water with a flavoring or coloring agent. An aerosol preparation can consist of a solution or suspension of the compound in a liquid carrier such as water, ethanol or glycerin; whereas in a powder dry aerosol, the preparation can include e.g., a wetting agent.

Formulations of the present invention comprise an active ingredient together with one or more acceptable carrier(s) thereof and optionally any other therapeutic ingredient(s). The carrier(s) should be “acceptable” in the sense of being compatible with the other ingredients of the formulation and not deleterious to the recipient thereof.

The pharmaceutical compositions of this invention may be orally administered in any orally acceptable dosage form including, but not limited to, capsules, tablets, and aqueous suspensions or solutions. In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the pharmaceutical compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

The pharmaceutical compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum.

The pharmaceutical compositions of this invention may also be administered by nasal aerosol or inhalation. Such compositions are prepared according to techniques well known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents known in the art.

It will be recognized by one of skill in the art that the form and character of the pharmaceutically acceptable carrier or diluent is dictated by the amount of active ingredient with which it is to be combined, the route of administration, and other well-known variables.

In a preferred embodiment, a composition used according to this invention is formulated for oral administration.

The above-described compounds and compositions are also useful in therapeutic applications relating to certain diseases. Methods according to this invention could be employed in discovering, developing, or implementing therapies for IL-1 or IL-18 mediated diseases.

These diseases include, but are not limited to, ischaemic stroke, including stroke-induced inflammation [Zaremba and Losy, 2003]; malaria [Nagamine et al., 2003]; acute myocardial infarction compared with unstable angina. [Yamaoka-Tojo, 2003]; Type-2 diabetes patients [Aso, 2003]; breast cancer patients [Gunel et al., 2003]; acute pancreatitis [Endo, 2001; Wereszczynska et al, 2002]; obesity and glucose intolerance [Olusi et al., 2003]; HIV [Ahmad et al, 2002]; disease progression in HIV-1 patients [Stylianou et al., 2003]; proatherogenicityin artherosclerosis [Elhage R et al., 2003]; murine atopic dermatitis model [Tsukuba and Yamamoto, 2003]; atopic dermatitis [Yoshizawa et al., 2002]; type-2 diabetes [Moriwaki, 2003]; celiac disease [Merendino et al., 2003]; psoriasis [Gangemi et al., 2003]; moderate-severe depression patients [Merendino, 2002]; lethality in sepsis patients [Emmanuilidis et al., 2002]; Behcet's disease [Hamzaoui et al., 2002]; systemic juvenile idiopathic arthritis and Still's syndrome [Kawashima et al]; systemic lupus erythematosus [Robak et al., 2002; Amerio et al., 2002]; metastatic breast cancer (vs. non-metastatic patients) [Gunel et al., 2002]; myasthenia gravis patients [Jander and Stoll, 2002]; CAD (coronary artery disease patients) is a strong independent predictor of death [Blankenberg et al., 2002]; IBD (inflammatory bowel disease) patients [Furuya et al., 2002]; Cushing's syndrome [Kristo et al., 2002]; fulminant hepatic failure patients [Yumoto et al., 2002]; CHF (congestive heart failure) patients [Seta et al., 2000]; Hep-C [Jia et al, 2003]; allergic rhinitis [Ariano et al., 2003]; obesity (especially in women after lifestyle changes and weight loss) [Esposito et al., 2003]; rheumatoid arthritis [Bresnihan et al, 2002]; Crohn's disease; asthma and other airway inflammatory diseases; and autoinflammatory diseases (such as Muckle-Wells syndrome). Other IL-1 or IL-18 mediated-diseases have been described (see, e.g., WO 95/35308, WO 97/22619, WO 99/47545, WO 01/90063, WO 04/058718, and WO 04/002961). This invention could also be used to evaluate treatments where IL-1 or IL-18 inhibition is contraindicated.

This invention also relates to a therapeutic method for treating certain diseases by (1) inhibiting IL-18 release from cells and/or (2) preventing the untoward, toxic or lethal effects of excessively high tissue levels of IL-18 in a mammal, including a human. This method comprises administering to a mammal an effective ICE inhibiting quantity of one or more compounds. This method also can be used for the prophylactic treatment or prevention of certain diseases amenable thereto. The invention provides a method for the treating these disorders by administering to a mammal, including a human, in need thereof an effective amount of such compounds.

The compounds, by inhibiting ICE and blocking the release of IL-18 or decreasing IL-18 levels and activity, as well as the pathophysiologic actions of excessive levels of IL-18 in each of these circumstances, directly facilitate the arrest or resolution of certain diseases, and facilitates the restoration of normal function. Together, these actions relate their novel use in treating certain diseases.

ICE inhibition may be measured by methods known in the art and as described more fully herein.

The phrase “inhibiting IL-18” means: a) a decrease of in vivo IL-18 levels in a mammal such as a human; b) a down regulation of IL-18 levels; or c) a down regulation of IL-1 activity, by inhibition of the direct synthesis of IL-1β or a post-translation event in vivo or in vitro.

The compounds may be useful in inhibiting the release of IL-18 release by monocytes, macrophages, neuronal cells, epithelial cells, endothelial cells, epidermal cells, mesenchymal cells (for example: fibroblasts, skeletal myocytes, smooth muscle myocytes, cardiac myocytes) and many other types of cells.

The term “condition” or “state” refers to any disease, disorder, or effect that produces deleterious biological consequences in a subject.

The level of IL-18 protein in the blood or cell of a patient or a cell culture (i.e., within the cell or the cell culture media) can be determined by, for example, assaying for immunospecific binding to IL-18 or to other proteins known to be produced as a result of the presence of active IL-1b. Such methods are known in the art. For example, immunoassays which can be used include, but are not limited to competitive and non-competitive assay systems, western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays and FACS analysis with labeled antibodies. Such assays well known in the art (see, e.g., Ausubel et al, eds., 1994, Current Protocols in Molecular Biology, Vol. 1, John Wiley & Sons, Inc., New York, which is incorporated by reference herein in its entirety).

Competitive binding assays can also be used to determine the level of IL-18. One example of a competitive binding assay is a radioimmunoassay comprising the incubation of labeled proteins from cells expressing IL-18 (e.g., ³H or ¹²⁵I) with an IL-18 antibody in the presence of increasing amounts of unlabeled IL-18, and the detection of the IL-18 antibody bound to the labeled IL-18. The affinity of the antibody of interest for a particular antigen and the binding off-rates can be determined from the data by Scatchard plot analysis. Competition with a second antibody can also be determined using radioimmunoassays. In this case, the antigen is incubated with antibody of interest conjugated to a labeled compound (e.g., ³H or ¹²⁵I) in the presence of increasing amounts of an unlabeled second antibody.

IL-18 levels can also be assayed by activity, for example, IL-18 levels can be assayed by a cell line that is capable of detecting bioactive levels of cytokines like IL-18 or a growth factor. According to one embodiment, the level of bioactive IL-18 in a biological sample is detected by incubating a cell line genetically engineered with isopropyl-b-D-thiogalactopyranoside. The cell line is incubated with the sample to be tested and cell death in the cell line is monitored by determining the intensity of blue color, which is indicative of a bioactive cytokine or growth factor in the sample tested. [See also, e.g., X.-S. Liu, Burns 20(1), pp. 40-44 (1994) for TNF].

A preferred method for measuring IL-18 levels in vivo is described below in the Examples.

Dosage levels in a pharmaceutical composition of this invention between about 0.01 and about 100 mg/kg body weight per day, preferably between about 0.5 and about 75 mg/kg body weight per day and most preferably between about 1 and about 50 mg/kg body weight per day of the active ingredient compound are useful in a monotherapy.

Typically, the pharmaceutical compositions of this invention will be administered from about 1 to 5 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Preferably, such preparations contain from about 20% to about 80% active compound.

Compositions of this invention may comprise a combination of active ingredients. When the compositions of this invention comprise a combination of a compound of this invention and one or more additional therapeutic agents, both the compound and the additional agent should be present at dosage levels of between about 10% to about 80% of the dosage normally administered in a monotherapy regime.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained. When the symptoms have been alleviated to the desired level, treatment should cease. Patients may, however, require intermittent treatment on a long-term basis upon any recurrence or disease symptoms.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of active ingredients will also depend upon the particular compound and other therapeutic agent, if present, in the composition.

Accordingly, a method for treating or preventing a disease of this invention in a subject comprises the step of administering to the subject any compound, pharmaceutical composition, or combination described herein.

In a preferred embodiment, the invention provides a method of treating a mammal, having one of the aforementioned diseases, comprising the step of administering to said mammal a pharmaceutically acceptable composition described above. In this embodiment, if the patient is also administered another therapeutic agent, it may be delivered together with the compound of this invention in a single dosage form, or, as a separate dosage form. When administered as a separate dosage form, the other therapeutic agent may be administered prior to, at the same time as, or following administration of a pharmaceutically acceptable composition comprising a compound of this invention.

The methods for identifying a compound or composition for treating a disease according to this invention include methods for screening of a plurality of compounds or compositions for their ability to inhibit ICE. According to one embodiment of this invention, high throughput screening can be achieved by having cells in culture in a plurality of wells in a microtiter plate, adding a different compound or composition to each well and comparing the ICE inhibition and/or IL-1β and/or IL-18 levels and/or activity in each cell culture to the levels or activity present in a cell culture in a control well. Controls that are useful for the comparison step according to this invention include cells or subjects that have not been treated with a compound or composition and cells or subjects have been treated with a compound or composition that is known to have no effect on ICE inhibition or activity. According to one embodiment of this invention, the high throughput screening is automated so that the steps including the addition of the cells to the plate up to the data collection and analysis after addition of the compound or composition are done by machine. Instruments that are useful in the comparison step of this invention, e.g., instruments that can detect labeled objects (e.g., radiolabelled, fluorescent or colored objects) or objects that are themselves detectable, are commercially available and/or known in the art. Accordingly, compounds and compositions according to this invention that are useful for treating the certain disease disclosed herein can be quickly and efficiently screened and evaluated.

All applications, patents, and references disclosed herein (above and below) are incorporated by reference. In order that this invention be more fully understood, the following preparative and testing examples are set forth. These examples are for the purpose of illustration only and are not to be construed as limiting the scope of the invention in any way.

### EXAMPLES

### Example 1

Tablet Formation

The composition of Compound A tablets used in the Examples below is provided in Table 1. The drug product was formulated to provide 300 mg of Compound A per tablet.

TABLE 1
Composition of Compound A 300 mg Tablets

| | Quantity | |
|---|---|---|
| Component | (mg/tablet) | Function |
| Compound A | 300 | Active |
| | | Ingredient |
| Microcrystalline | 277.50 | Filler |
| Cellulose (NF) | | |
| Pregelatinized Starch | 131.25 | Disintegrant |
| (NF) | | |
| Sodium Starch Glycolate | 15.00 | Disintegrant |
| (NF) | | |
| Colloidal Silicon Dioxide | 11.25 | Glidant |
| (NF) | | |
| Talc (USP) | 7.50 | Glidant |
| Magnesium Stearate (NF) | 7.50 | Lubricant |
| Total | 750 | |

### Example 2

Compound A Administration

Compound A was evaluated in a double-blind, randomized, placebo-controlled, oral dose, sequential group study in healthy male subjects. 9 subjects received 900 mg Compound A 14 days. 3 subjects received placebo treatment. Doses were administered three times a day at 8-hour intervals on Days 1 to 13 inclusive, and once in the morning of Day 14. All doses were administered in the fasted state.

### Example 3

Blood Sampling Procedure:

Blood samples (1×3.5 mL) were taken by venepuncture or cannulation of a forearm vein(s).

Blood samples were collected into 3.5 mL SST Vacutainer® tubes (Becton Dickinson UK Ltd., Oxford) and, after mixing, stored at ambient temperature for at least 30 min prior to centrifugation. The samples were centrifuged, within 1 hour of collection, at 1500 g for 10 minutes at approximately 4° C. For each sample, the separated serum was transferred into two 5 mL suitably labelled polypropylene tubes (at least 0.6 mL in each tube), and stored within 2 hours of collection, at approximately −70° C. the laboratory for IL-18 assay.

### Example 4

IL-18 Assay Procedure:

IL-18 was assayed using a sandwich IL-18 ELISA technique (Human IL-18 ELISA Kit, Medical and Biological Laboratories, Nagoya, Japan). Standards, Controls and Samples were incubated in wells coated with an anti-human IL-18 monoclonal antibody. After washing, a peroxidase conjugated anti-human IL-18 monoclonal antibody was added to the well and then re-incubated. After another washing, a substrate reagent was then added to the well. After further incubation the reaction was stopped by the addition of an acid solution. The developed color was then measured at an OD of 450 using a 630 nm reference filter.

Compound A was shown to inhibit IL-18 levels in vivo. Over the 14-day treatment period, Compound A led to a gradual but marked inhibition of serum IL-18 concentrations, whereas the serum IL-18 concentration in placebo subjects remained essentially unchanged.

The median baseline-normalized concentration of serum IL-18 gradually decreased over the 14-day treatment duration for Compound A dosing, whereas the median levels for subjects on placebo remained essentially unchanged. The range of t_{max,IL-18} and t_{min,IL-18} parameters for the 900 mg q8 h treatment indicate that the maximum serum IL-18 concentration for all subjects receiving this treatment occurred at the pre-dose timepoint, and the minimum concentration for all subjects occurred on Day 14.

Other ICE and IL-18 related assays are described in detail in U.S. Pat. No. 5,985,863, which is incorporated herein by reference (see, e.g., Examples 1-6).

While we have described a number of embodiments of this invention, it is apparent that our basic examples may be altered to provide other embodiments, which utilize the compounds and methods of this invention. Therefore, it will be appreciated that the scope of this invention is to be defined by the appended claims rather than by the specific embodiments, which have been represented by way of example.

1. A method for evaluating whether an ICE inhibitor is effective in ameliorating, treating or preventing an IL-1 mediated condition and/or an IL-18 mediated condition, the method comprising:
measuring IL-18 levels in the blood of patients before treatment,
and further measuring IL-18 levels in the blood of patients after treatment,
wherein a decrease in IL-18 levels after treatment is indicative of effectiveness.

2. A method for evaluating whether a formulation comprising an ICE inhibitor is effective in ameliorating, treating or preventing an IL-1 mediated condition and/or an IL-18 mediated condition, the method comprising:
measuring IL-18 levels in the blood of patients before treatment with said formulation,
and further measuring IL-18 levels in the blood of patients after treatment with said formulation,
wherein a decrease in IL-18 levels after treatment is indicative of effectiveness.

3. A method for evaluating whether a dosage amount or regime of an ICE inhibitor is effective in ameliorating, treating or preventing an IL-1 mediated condition and/or an IL-18 mediated condition, the method comprising:
measuring IL-18 levels in the blood of patients before treatment with said dosage amount or regime,
and further measuring IL-18 levels in the blood of patients after treatment with said dosage amount or regime,
wherein a decrease in IL-18 levels after treatment is indicative of effectiveness.

4. A method of determining whether a patient is a candidate for therapy with an ICE inhibitor, comprising determining IL-18 levels in the subject, comparing IL-18 levels in the subject with IL-18 levels in a normal individual, wherein higher IL-18 levels in the potential subject qualifies the patient for therapy.

5. A method for predicting the therapeutic outcome of an ICE inhibitor therapy, comprising determining IL-18 levels in the subject, prior to and after administration of the ICE inhibitor, wherein a decrease in IL-18 levels after administration of the ICE inhibitor is predictive of a potentially successful therapeutic outcome.

6. A method for identifying a compound that ameliorates, treats, or prevents an IL-1 mediated disease, the method comprising:
measuring IL-18 levels in a subject prior to administration of the compound,
and further, measuring the levels after administration of the compound,
wherein a decrease in the IL-18 levels after administration of the compound indicates the compound may ameliorate, treat, or prevent the IL-1 mediated condition or disease.

7. A method for identifying a compound that ameliorates, treats, or prevents an IL-18 mediated disease, the method comprising:
measuring IL-18 levels in a subject prior to administration of the compound,
and further, measuring the levels after administration of the compound,
wherein a decrease in the IL-18 levels after administration of the compound indicates the compound may ameliorate, treat, or prevent the IL-18 mediated condition or disease.

8. The method of any one of claims 1-3, wherein the ICE inhibitor is selected from the group consisting of ICE inhibitors of any one the compounds of WO 04/058718, WO 04/002961, WO 03/088917, WO 03/068242, WO 03/042169, WO 98/16505, WO 93/09135, WO 00/55114, WO 00/55127, WO 00/61542, WO 01/05772, WO 01/10383, WO 01/16093, WO 01/42216, WO 01/72707, WO 01/90070, WO 01/94351, WO 02/094263, WO 02/42278, WO 03/106460, WO 03/103677, WO 03/104231, U.S. Pat. No. 6,184,210, U.S. Pat. No. 6,184,244, U.S. Pat. No. 6,187,771, U.S. Pat. No. 6,197,750, U.S. Pat. No. 6,242,422, April 2001 American Chemical Society (ACS) meeting in San Diego, Calif., USA., WO 02/22611, U.S. 2002/0058630, WO 02/085899, WO 95/35308, WO 97/22619, WO 99/47545, and WO 01/90063.

9. The method of any one of claims 1-3, wherein the compound is a caspase inhibitor, an IL-1 inhibitor, or an IL-18 inhibitor.

10. The method of any one of claims 1-3 wherein the compound is selected from a compound of WO 95/35308, WO 97/22619, WO 99/47545, or WO 01/90063.

11. The method of any one of claims 1-3 wherein the compound is selected from a compound of WO 99/47545 or WO 01/90063.

12. The methods of any one of claims 1-3 wherein the compound is: 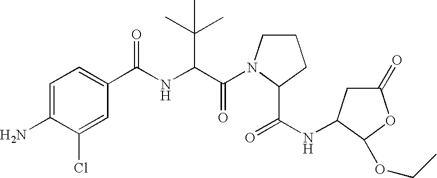
and each stereoisomer thereof, including:

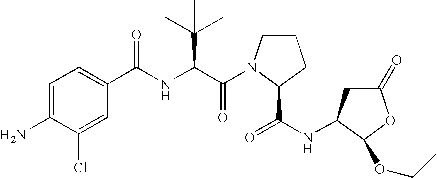

13. The methods of any one of claims 1-3 wherein the compound is: 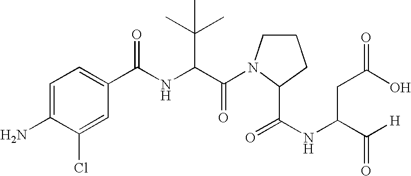
and each stereoisomer thereof, including:

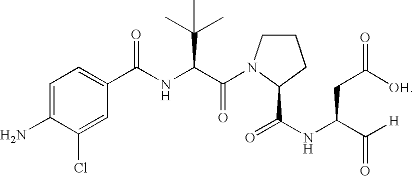

14. A pharmaceutical composition for ameliorating, treating, or preventing a certain disease in a subject, comprising a compound selected or evaluated according to a method of this invention and a pharmaceutically acceptable carrier.

15. A method of following the course of therapy with an ICE inhibitor comprising the step of monitoring the levels of IL-18 in the patient at the beginning and during continuation of therapy.
